(19) United States
(12) Patent Application Publication
(10) Pub. No.: US 2025/0129486 A1
(43) Pub. Date: Apr. 24, 2025

(54) A METHOD FOR MANUFACTURING PHARMACEUTICAL GRADE HYPOCHLOROUS ACID

(71) Applicant: THOCLOR LABS (PTY) LTD, Lynedoch, Stellenbosch (ZA)

(72) Inventor: Hendrik Christoffel ROOS, Lynedoch, Stellenbosch (ZA)

(21) Appl. No.: 18/835,170

(22) PCT Filed: Feb. 10, 2023
(86) PCT No.: PCT/IB2023/051201

§ 371 Date: Aug. 1, 2024

(30) Foreign Application Priority Data
Feb. 10, 2022 (GB) 2201705.7

(51) Int. Cl. C25B 1/26; A61K 33/00; C25B 9/17; C25B 11/063; C25B 11/081; C25B 15/031

(52) U.S. Cl. CPC C25B 1/26; A61K 33/00; C25B 9/17; C25B 11/063; C25B 11/081; C25B 15/031

(57) ABSTRACT

The present invention relates to a method of manufacturing pharmaceutical grade hypochlorous acid. The hypochlorous acid is produced in an electrolysis chamber 30 at a pH of around 2.0 using a 6% platinum grade hydrochloric acid 36, and pharmaceutical grade water 22. The pH of the produced hypochlorous acid is adjusted using platinum grade disodium hydrogen orthophosphate a buffering agent to a pH of between 4.0 and 5.8.

### INTRODUCTION

The present invention relates to a method of manufacturing pharmaceutical grade hypochlorous acid. In particular, hypochlorous acid may be produced in an electrolysis chamber at a pH of around 2.0 using a platinum grade hydrochloric acid, preferably 6% platinum grade hydrochloric acid and pharmaceutical grade water. The pH of the produced hypochlorous acid may be adjusted using a buffering agent, preferably platinum grade disodium hydrogen orthophosphate to a pH of between 4.0 and 5.8.

### BACKGROUND TO THE INVENTION

HOCl is the most effective disinfectant in the chlorine family and should be the only free chlorine molecule present in the HOCl solution if the solution is to be applied in the pharmaceutical environment. HOCl is 80 to 120 times more effective as a disinfectant than the hypochlorite ion (OCl⁻). In addition, because HOCl has no charge and has a low molecular weight, it is more effective than the other chlorine molecules to penetrate the cell walls of pathogens. It also reacts more rapidly than other chlorine-based disinfectants to oxidation reactions with organic matter, being the critical components of microbial cells.

Conversely, the hypochlorite ion is a poor disinfectant because of its inability to diffuse through the cell wall. Hypochlorite ion carries a negative charge, it is electrostatically repelled from the cell walls, which are also negatively charged.

HOCl has advantages over sodium hypochlorite (NaOCl) and other chlorine-containing molecules in that within its effective antimicrobial concentration range, it is non-irritating, non-sensitizing and has no cytotoxicity to mammalian cells. Apart from its effect on pathogens, HOCl also has anti-inflammatory properties and destroys biofilm.

### HOCl is Commonly Produced Using Electrolysis of Salt.

The electrolysis of salt method for producing HOCl is not suitable for producing pharmaceutical grade HOCl solution. The reasons follow below.

2Cl⁻+2e⁻→Cl₂,followed by Cl₂+H₂O→HOCl+HCl. Anode reaction:

H₂O+2e⁻→H₂+2OH⁻ Cathode reaction:

The combined reaction in the two-chamber electrolytic bath of the salt electrolysis method is:

2NaCl+2H₂O→2NaOH+HOCl+H₂+Cl₂ [Reaction Formula 1]

A two-chamber electrolytic bath separates the catholyte (mostly NaOH, H₂) from the anolyte (mostly Cl₂, HOCl, HCl). The anolyte is mostly used as disinfectant as it also contains varying quantities of disinfection by-products. Anolyte has been advocated for use in wounds, despite the potential presence of disinfection by-products at therapeutic concentrations of HOCl (>200 mg/L).

Disinfection by-Products Resulting from the NaCl Electrolysis Method of Producing HOC.
a) Sodium hypochlorite (NaOCl) is formed through the buffering of HCl with NaOH in an acidic anolyte solution to raise the pH-level from the manufactured pH (2.0 to 3.0) to pH 4.0 to 5.80. The buffered pH of the solution is to ensure that HOCl will be the prevalent free chlorine species in the solution.

HOCl+NaOH→NaOCl+H₂O [Reaction Formula 2]
b) Sodium chlorite (NaClO₂) is formed through the electrolysis of NaCl.

NaCl+O₂→NaClO₂ [Reaction Formula 3]
c) Sodium chlorate (NaClO₃) is formed through the electrolysis of NaCl.

NaCl+3H₂O+6e⁻→NaClO₃+3H₂ [Reaction Formula 4]
Sodium hypochlorite (NaOCl), sodium chlorite (NaClO₂) and sodium chlorate (NaClO₃) are potential toxic molecules that prove that the electrolysis of NaCl is not suitable to produce pharmaceutical grade HOCl and is not suitable for pharmaceutical application.

It is accordingly an object of the invention to provide a method for manufacturing pharmaceutical grade hypochlorous acid, in particular that reduces or prevents the formation of disinfection by-products.

### SUMMARY OF THE INVENTION

According to a first aspect of the invention there is provided a method of manufacturing pharmaceutical grade hypochlorous acid, the method comprises:
a) adding platinum grade hydrochloric acid with a concentration of 5 to 7%, to an inlet of an electrolysis chamber;
b) executing an electrolysis process to produce hypochlorous acid;
c) supplying water with a conductivity of 0.15 to 0.25 μS/cm (microSiemen/cm) to mix with and produce hypochlorous acid with a pH of 1.5 to 2.5;
d) adding a buffering agent to the hypochlorous acid produced at step c) to adjust the pH of the hypochlorous acid to between 4.0 and 5.8, producing a pharmaceutical grade hypochlorous acid.

Typically, the hypochlorous acid produced at step c) has a concentration of 350 to 400 mg/L.

Preferably, the buffering agent is added to the hypochlorous acid in less than 2 seconds, typically within 1 second of the production of the hydrochloric acid.

The pH of the hypochlorous acid produced at step c) is typically about 2.0.

The platinum grade hydrochloric acid typically has a concentration of 6%.

Preferably, the buffering agent is an orthophosphate buffer in an aqueous solution, preferably platinum grade disodium hydrogen orthophosphate or dipotassium hydrogen orthophosphate buffer in an aqueous solution at a 1% to 10% m/m concentration.

The pH of the hypochlorous acid in step d) may be about 5.40.

Typically, the platinum grade hydrochloric acid is added to the electrolysis chamber at a rate of between 1-5 mL/min, optionally between 1.5-4.5 mL/min, optionally between 2-4 mL/min.

Typically, the pharmaceutical grade water is added to the manufactured hypochlorous acid at the outlet of the electrolysis chamber at a rate of between 0.3 to 2 L/min, optionally between 0.6-1.8 L/min, optionally between 1.0 to 1.5 L/min.

The water at step c) typically has a conductivity of 0.18 to 0.22 μS/cm, preferably less than 0.21 μS/cm.

The electrolysis chamber may be a single chamber reactor.

The electrolysis chamber may comprise of multiple electrolytic plates.

The electrolytic plates are typically iridium oxide-coated titanium.

The buffering agent may be added to the hypochlorous acid in a pH correction mixing chamber.

Preferably, the pH of the hypochlorous acid is adjusted within one second in the pH correction chamber.

Preferably, he pH correction chamber consists of a separate entry point for hypochlorous acid and the buffering agent.

The pH correction process may further comprise a dosing pump that automatically buffers the hypochlorous acid to the adjusted pH.

Preferably, the mixing chamber includes a pH measuring electrode for real-time pH-measurement.

Typically, the pH electrode communicates with a controller that adjusts buffer dosing through a dosing pump to deliver a pre-determined product pH.

The dosing pump may be controlled by a proportional-integral-derivative controller (PID controller).

The pH correction chamber may comprise of an augur screw mixer for mixing the hypochlorous acid and the buffering agent.

Any hydrogen chloride produced during electrolysis is removed from the solution of hypochlorous acid/hydrogen chloride during step d) by the buffering agent.

The invention also covers a pharmaceutical grade hypochlorous acid produced by the method according to any of claims 1 to 19.

According to another embodiment of the invention, there is provided a method for treating inflammation and infections from fungi, bacteria or viruses The method comprises administering a therapeutically effective amount of the pharmaceutical grade hypochlorous acid described above to a subject in need thereof.

According to another embodiment of the invention, there is provided a pharmaceutical grade hypochlorous acid described above for use in a method for treating inflammation and infections from fungi, bacteria, or viruses, wherein the method comprises of administering a therapeutically effective amount of said pharmaceutical grade hypochlorous acid described above to a subject in need thereof.

According to another embodiment of the invention, there is provided the use of the pharmaceutical grade hypochlorous acid described above in the manufacture of a medicament for treating inflammation, infections from fungi, bacteria or viruses, wherein the method comprises of administering a therapeutically effective amount of said medicament to a subject in need thereof.

The Infections may include:
1) skin afflictions resulting from:
   a) inflammageing which refers to long-term low-grade inflammation that leads to ageing of the skin and chronic inflammatory skin conditions such as rosaceae, acne, eczema, and folliculitis barbae,
   b) aftercare that treats inflammation resulting from invasive skin treatment such as skin laser (ablative and non-ablative), skin needling (also called mesoderm treatment), injection of platelet rich plasma or dermal fillers, skin scraping, dermabrasion, the removal of cosmetic tattoo through laser or chemical means,
   c) Acne keloidaris nuchae, infection with inflammation that occur on the back of the neck, and
   d) Nappy rash, omphalitis, and cradle cap infections in babies,
2) Eye infections caused by bacteria, viruses, and fungi, such as meibomianitis, conjunctivitis, uveitis, and keratitis,
3) Sinusitis,
4) Vaginal infections,
5) Allergic and inflammatory skin conditions,
6) Insect bites or allergic reaction to antigens (pollen, plant, or animal material),
7) Atopic dermatitis, psoriasis,
8) Sunburn, and
9) Wounds.

The fungi may be selected from:
a) Fungal nail infections including yeast onychomycosis,
b) Athlete's foot (tinea pedis), fungal infection of the groin (tinea cruris),
c) Candidiasis of the oral cavity such as chronic mucocutaneous candidiasis, mucosal candidiasis, and cutaneous candidiasis,
d) Folliculitis,
e) Aspergillosis, cryptococcosis, histoplasmosis, blastomycosis, paracoccidioidomycosis, mucormycosis, oral geotrichosis, or fusariosis,
f) Candida fungal infections of the breast nipple,
g) oral fungal infections in babies, and
h) Infective lung conditions, viral e.g., caused by severe acute respiratory syndrome (SARS).

The bacteria may be selected from:
a) Erysipelas caused by streptococcus,
b) Bacterial folliculitis caused by Pseudomonas aeruginosa,
c) Furunculosis, carbuncle and impetigo caused by Staphylococcus aureus,
d) Erythrasma, caused by Corynebacterium minutissimum,
e) Periorbital bacterial infections, which could contribute to dry eye syndrome,
f) Methicillin-resistant Staphylococcus aureus (MRSA) skin infections,
g) Leprosy, through the bactericidal action on Mycobacterium leprae,
h) Multiple drug resistant bacteria such as extreme drug resistant and drug sensitive Mycobacterium tuberculosis, Pseudomonas aeruginosa, Acinetobacter baumannii, carbapenem resistant enterobacteriaceae (CRE), incl. OXA-48 Klebsiella pneumoniae, Candida parapsilosis, and human coronavirus,
i) Infective lung conditions, bacterial, including acute bacterial pneumonia, chronic lung conditions due to primary and secondary bacterial infection e.g., Pseudomonas aeruginosa,
j) Infective lung conditions with a strong inflammatory component like tuberculosis (drug sensitive as well as drug resistant strains of Mycobacterium tuberculosis), and lung affliction in HIV patients, as well as severe acute respiratory syndrome resulting from respiratory virus infection,
k) Infective fungal lung conditions, candida and mucormycosis (black fungus), respiratory syncytial virus lung infections.
l) Treatment of degenerative lung conditions to prevent secondary infection. Conditions include cystic fibrosis, chronic obstructive pulmonary disease (COPD) and pulmonary fibrosis,
m) Treatment of degenerative lung conditions to reduce scarring (COPD, atelectasis, post infective lung fibrosis),
n) Treatment of inflammatory lung conditions (asthma, HIV), pneumonitis,
o) Chancroid, genital herpes, granuloma inguinale and lymphogranuloma venereum.

The infections may be combinations of bacterial and fungal infections, such as biofilm, e.g., acne, folliculitis barbae and perioral dermatitis.

Wounds may be selected from:
a) Acute wounds such as traumatic, surgical and burn wounds,
b) Chronic wounds, including venous ulcers, diabetic foot ulcers, arterial ulcers, rheumatoid ulcers, pressure sores, or any non-healing wound,
c) Orthopaedic periprosthetic joint infection resulting from septic joint replacement,
d) Infected burn wounds,
e) Acute and chronic episiotomy wounds.

Inflammation may be selected from:
a) Inflammatory bowel disease or ulcers of the perianal area,
b) Ulcerative colitis.

The invention also covers a cosmetic method of rejuvenating the skin comprising administering the pharmaceutical grade hypochlorous acid described above to the facial, neck, chest, arms and hands skin of a subject.

### BRIEF DESCRIPTION OF THE DRAWINGS

FIG. 1 is a graph showing HOCl, Cl₂ and OCl⁻ exist in an equilibrium which is pH dependent;

FIG. 2 is a process flow diagram for manufacturing pharmaceutical grade HOCl;

FIG. 3 is a graph showing spectrophotometric analysis of a solution that contains low levels of hypochlorous acid, toxic chlorate, and a strong chloride signal, indicating that the solution was probably manufactured through the electrolysis of salt (NaCl); and

FIG. 4 is a graph showing spectrophotometric analysis of a solution containing a strong signal of pharmaceutical grade hypochlorous acid. This analysis does not demonstrate any signal for the presence of any toxic substances and is an example of a solution manufactured through the method described in this Invention.

### DESCRIPTION OF AN EMBODIMENT OF THE INVENTION

The term pharmaceutical grade refers to a standard of purity suitable for use as a medicine. The method of producing a solution of pharmaceutical grade hypochlorous acid, particularly of high concentration (above 250 mg/L) and at the correct pH where only HOCl chlorine species is present in the solution, has not been described before. Furthermore, the solution containing hypochlorous acid should demonstrate storage stability.

The present invention relates to a method of manufacture of pharmaceutical grade hypochlorous acid (HOCl) solution. The solution is free of toxic by-products and is manufactured by means of a process that is novel. Furthermore, the manufacturing process results in high concentration HOCl solution at a pre-determined pH level. The solution is stable when kept in UV-protected glass for two years. Due to the method and process of manufacture, the solution is of a pharmaceutical grade.

The present invention uses an electrolyte of platinum grade hydrochloric acid, pharmaceutical grade purified water, and platinum grade disodium hydrogen orthophosphate as a real-time buffering and stabilizing agent.

The hypochlorous acid is manufactured by means of electrolysis (hydrolysis) of the hydrochloric acid in a mono chamber electrolyte bath that contains an electrode. The electrolyte is fed to the electrolytic bath via an acid pump and water is added to the high concentration chlorine solution that results from the electrolysis process at a specific flow rate to produce a pre-determined concentration of free chlorine solution. Real-time buffering of the manufactured low pH (high acidity) free chlorine solution through an automated buffering system consisting of a pH measuring probe and a dosing pump, controlled by a proportional integral derivative controller (PID Controller), which automatically and in real time buffers the solution with an aqueous solution of platinum grade disodium hydrogen orthophosphate to the correct pre-determined pH-value. Real-time buffering prevents the solution from contamination by disinfection by-products that result from the decomposition and dissociation of hypochlorous acid.

### Electrolysis of Hydrochloric Acid (HCl) to Form HOCl

The manufacturing of HOCl by means of electrolysis of HCl holds many advantages over the method described in the background of the invention and is the first leg of the present invention. The principal advantage of a solution of HCl as electrolyte is that the hypochlorous acid solution is sodium free. HOCl that is manufactured using HCl will not lead to the synthesis of toxic components, more specifically sodium hypochlorite (NaOCl), sodium chlorite (NaClO₂) and sodium chlorate (NaClO₃).

Where NaCl electrolysis requires a two-chamber electrolytic bath to separate the acidic anolyte from the alkaline catholyte, electrolysis of HCl is done in a single chamber.

At the cathode, hydrogen generation occurs.

2H₂O→2H₂+4OH⁻ [Reaction Formula 5]

At the anode, an oxygen generation reaction occurs.

2H₂O→O₂+4H⁺ [Reaction Formula 6]

The anode also oxidises the chlorine of the HCl solution.

2Cl⁻→Cl₂+2e [Reaction Formula 7]

Cl₂ generated at the anode is hydrolysed.

Cl₂+H₂O→HOCl+HCl [Reaction Formula 8]

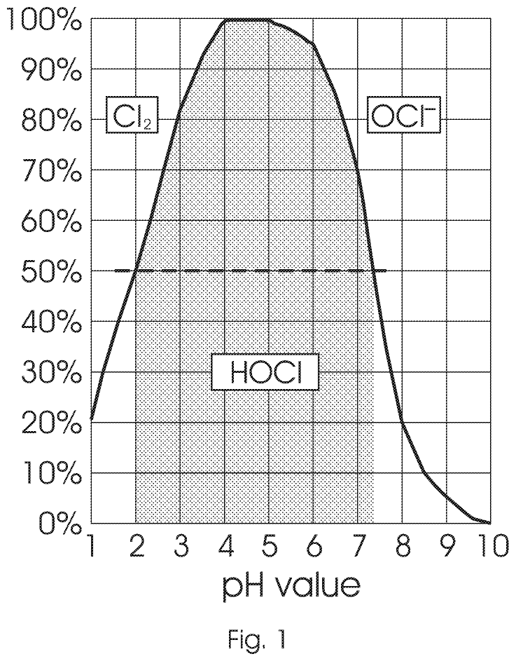

This reaction is responsible for the formation of HOCl and HCl in equal quantities with resultant low pH (s 2.0) of the solution. HCl is a much stronger acid (pKa −6.30) when compared to HOCl (pKa 7.53). The solution, therefore, due to its low pH, contains free chlorine of predominantly Cl₂ species (see FIG. 1).

### HOCl and the Effect of the pH of the Solution.

The most important chlorine specie reaction in the chlorination of an aqueous solution is the formation of hypochlorous acid.

Hypochlorous acid is a ‘weak’ acid, meaning that it tends to undergo partial dissociation to a hydrogen ion (H⁺) and a hypochlorite ion (OCl⁻).

HOCl [symbol] H⁺+OCl⁻ [Reaction Formula 9]

HOCl Dissociation from a Rise in pH of the Water.

In an alkaline environment, the following reaction occurs with water.

H₂O<H⁺+OH⁻ [Reaction Formula 10]

Since the tendency of these two ions (H⁺+OH⁻) to react and form H₂O is much stronger than the tendency of water to break down into the ions, an increase in pH results in fewer H⁺ ions and more OH⁻ ions.

The H⁺, released by the breakdown of HOCl (due to the presence of OH⁻) reacts to form water (H⁺+OH⁻═H₂O) and leaves behind residual OCl⁻ ions. Hypochlorite therefore becomes the dominant species in the solution.

HOCl Dissociation from a Drop in pH of the Water.

If the pH becomes more acidic and H⁺ ions become readily available again, the OCl⁻ ions revert to HOCl, which is the pathogen killing form of chlorine.

With further drop in pH, more H⁺ drives the reaction:
Cl₂+H₂O [symbol] HOCl+HCl to the left and Cl₂ becomes the dominant chlorine species in the solution.

Cl₂, HOCl and OCl⁻ exist in an equilibrium and the predominance of each of these chlorine species is therefore dependent on the pH of the water. At pH 4.0-5.80, all the chlorine is present as HOCl. In water with pH between 5.80 and 8.50, the reaction is incomplete and both species (H⁺+OCl⁻) are present to some degree. At pH 7.50, half of the total chlorine is present as HOCl and the other half as OCl⁻. The dissociated hypochlorite ion (OCl⁻) predominates at higher pH values above 7.50. At pH value of 10-14, all the chlorine to be present as OCl⁻ (see FIG. 1).

At low pH (pH 4.0 and lower) hydrolysis of Cl₂ is not complete, and a significant fraction remains in the form of molecular chlorine Cl₂.

Therefore, in an aqueous environment, the water pH will affect the chemistry of chlorine through its pH sensitivity; this is important as the pH value increases or decreases.

### Advantages of the Present Invention

### 1. Sodium-Free Manufacturing.

The electrolysis of HCl is a more suitable production method of HOCl, mainly because no sodium is introduced as electrolytic component [See Reaction Formulae 5, 6, 7 and 8]. This prevents the formation of sodium-containing disinfection by-products.

### 2. Pharmaceutical Grade Reactants.

Only pharmaceutical grade water, platinum grade HCl and platinum grade disodium hydrogen orthophosphate are used in the manufacturing process. Due to the high reactivity of HOCl, the presence of inorganic and organic molecules will lead to various potential toxic compounds being present in the solution.

Not only should no sodium be present during the electrolytic generation of HOCl, but it is also recommended to avoid inorganic and organic compounds that may contaminate the electrolysis process.

Some impurities that may exist in water include dissolved inorganic ions such as NO²⁻, SO³⁻, Cu²⁺, and CuS, which results in the rapid consumption of HOCl through oxidation reactions with these compounds. Chlorine-impurity reactions will create undesired by-products that are harmful to human health. The oxidation by HOCl will also reduce the concentration of HOCl, which will negatively affect the shelf life. Examples of HOCl oxidation ingredients are insoluble ferrous, trihalomethane, halo-acetic acid, mono-, di- and trichloramine and chlorophenols.

The utilization of platinum grade reactants and pharmaceutical grade water will ensure that none of these oxidation compounds will exist in the HOCl solution.

### 3. Producing a Stable Pharmaceutical Grade HOCl Solution.

Solutions containing HOCl are generally regarded to be unstable, demonstrating rapid decomposition of the HOCl, though a method to stabilize HOCl has been described (Pub. No.: US 2014/0134277 A).

The stability of HOCl solution is dependant of multiple factors.
i) The stability of HOCl solution requires shielding from light, and minimal air contact. The photolysis of HOCl is more pronounced with higher concentrations of HOCl (>80 mg/L) but does not seem to be more pronounced for higher concentrations of OCl⁻. The presence of light accelerates the exothermic decomposition into OH, Cl and O₃

HOCl+UV light→OH+Cl⁻+O₃ [Reaction Formula 11]
It is therefore essential to store solutions containing HOCl in UV-protected inert containers.
ii) The container in which the solutions are kept should be made of glass. In our experience there is marked decomposition of HOCl in any form of plastic container. Glass containers avoid oxidation reactions between the container and HOCl.
iii) Stable HOCl production requires platinum grade reactants, including pure water without any organic and inorganic compounds or ions, as the oxidation of these compounds will deplete the HOCl concentration in the solution.
iv) Correcting the solution pH to between 4.0-5.80 immediately after manufacturing, through real-time buffering. Variation outside pH 4.0 to 5.80 will not only result in the dissociation of HOCl into Cl₂ or OCl⁻ but will provide the environment for the decomposition of HOCl to form chlorate. It is therefore of utmost importance that this pH level

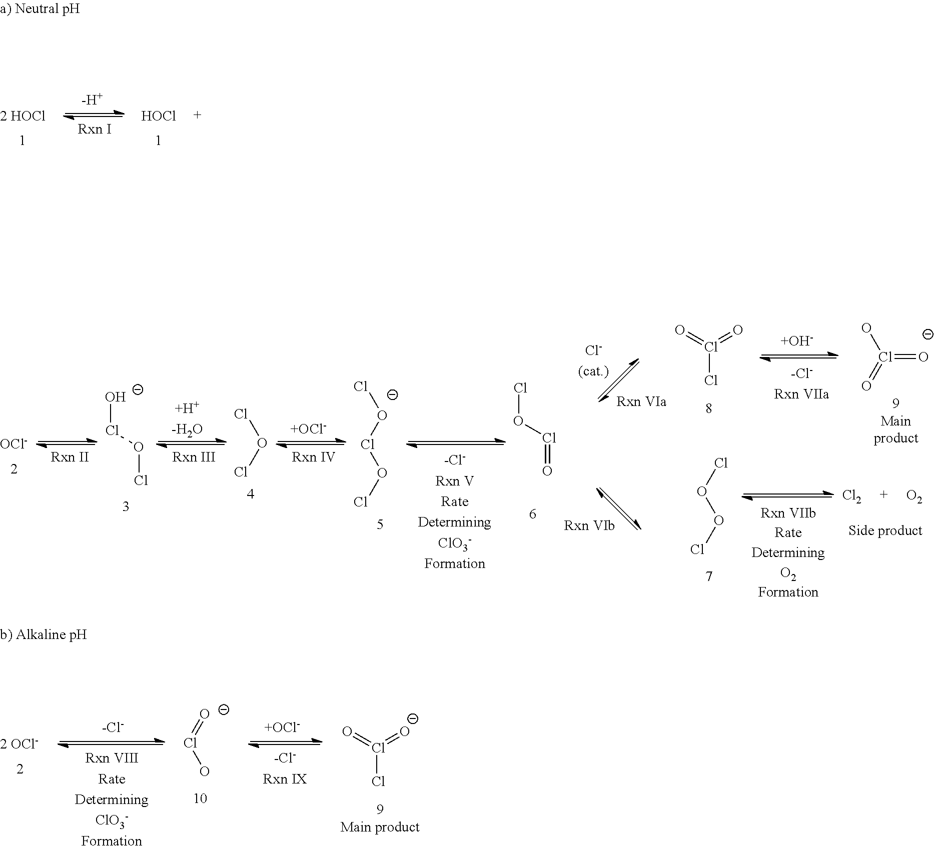

be raised through real-time buffering of the solution, so that the pH level is maintained between 4.0 to 5.8 before the solution enters the storage tank.

### Dissociation of HOCl Under Neutral, Alkaline, and Acidic pH Conditions.

As mentioned before, the rate of dissociation of HOCl depends on the pH of the water. If the pH is below 5.80 and above 4.0, no dissociation will occur and HOCl will be the only chlorine species present. When the pH is above 5.80, hypochlorite ions start to dissociate from the HOCl and in an acidic environment below pH 4.0 HOCl starts to dissociate to Cl₂.

Any amount of hypochlorite in the solution not only poses a risk of toxicity but will lead to the accelerated decomposition of HOCl. Hypochlorite ions plays a key role in the decomposition of HOCl.

Decomposition of HOCl to OCl⁻, Cl₂, and Chlorate Under Neutral, Alkaline, and Acidic Conditions.

Decomposition of HOCl and OCl⁻ proceed through chlorite (ClO₂) to chlorate (ClO₃) and oxygen (O₂) under neutral and alkaline conditions.

In a solution with pH above 5.80, HOCl dissociates progressively to OCl⁻ as the solution becomes more alkaline, the OCl⁻ becomes the substrate for the decomposition of HOCl to chlorate (See b) Alkaline pH reaction above).

Both are toxic substances that are not suitable to be present in pharmaceutical grade HOCl solutions.

### Acidic pH

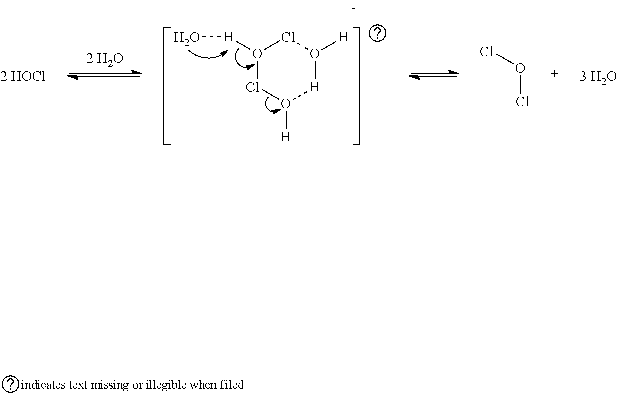

HOCl not only dissociates to Cl₂ in an acidic environment, but also decomposes to chlorate (ClO₃), even where OCl⁻ is completely present.

The deprotonation of HOCl (HOCl [symbol] OCl⁻+H⁺) provides the OCl⁻ needed for the reaction to be completed (See a) neutral pH and b) alkaline pH reaction above and acidic pH reaction above).

ClO₃ is a toxic substance that is not suitable to be present in pharmaceutical grade HOCl solutions.

### Real-Time Buffering of Acidic HOCl Solution.

The goal of a buffer is to keep the pH of a solution within a narrow range. In this case the pH needs to be at the pre-determined level of 5.40 (but generally between pH 4.0 to 5.80, as HOCl will be the only chlorine species present in the solution).

The presence of a strong acid such as HCl in water [Reaction Formula 8] results in the reaction

HCl+H₂O→H₃O⁺+Cl⁻ [Reaction Formula 12]

In other words, the proton (H⁺) from the acid binds to neutral water molecules to form H₃O⁺ raising the concentration of H⁺. The resulting higher concentration of (H⁺) makes the solution more acidic and leads to a drop in the pH of the solution. The stability of pharmaceutical grade HOCl solution that is manufactured by means of HCl electrolysis is dependent on the buffering process to remove enough H⁺ protons from the solution to raise the pH to within the pre-determined manufacture range. Equally important is what buffering agent will be used for this purpose.

### Buffering Agent

The choice of buffering agent is determined what conjugate acid will result from the buffering action of the buffering agent.

The conjugate acid of disodium hydrogen orthophosphate (Formula Na₂HPO₄ CAS 7758-79-4, anhydrous, extra pure, platinum range) is phosphoric acid.

Na₂HPO₄ or dipotassium hydrogen orthophosphate (K₂HPO₄) is the buffer of choice in the manufacturing process of pharmaceutical grade HOCl solution. Sodium may be introduced at the buffering stage of manufacturing as the electrolysis of HCl has been sodium free and therefore no disinfection by-products exist in the solution.

Orthophosphate (PO₄³⁻) is also called “phosphate” because it is very easy to bond with positive compounds since it has three “extra” electrons that strongly want to bond with protons. It therefore reacts fast with a strong acid (in this case HCl), allowing for real-time buffering of the strong acid HCl in the solution.

Disodium hydrogen orthophosphate (Na₂HPO₄) is a reasonably strong base (pH 8.50-9.60). It reacts with HCl to form its conjugate acid, phosphoric acid, and the salt sodium chloride (NaCl).

Na₂HPO₄+2HCl→H₃PO₄+2NaCl [Reaction Formula 13]

The selective buffering of the HCl in the HOCl solution raises the pH from circa pH 2.0 to the desired 5.40 in an expedient manner, immediately after the HOCl/HCl solution was manufactured. The expediency of the buffering process assures that no chlorate forms.

There are problems using other buffering agents such as magnesium carbonate. The total concentration of chlorine generated during the electrolysis process is affected by the by-reactions of chlorine with Mg2+ ions present in the solution. This leads to instability of the manufactured product. The drop in concentration is further exacerbated by decomposition of hypochlorous acid due to unfavourable pH range before and during buffering.

The applicant's method of real-time buffering leads to a slight rise in free chlorine concentration, when compared to postproduction buffering.

The applicant postulates that the presence of chlorine gas (Cl₂) in the solution leads to more free chlorine formation (Reaction Formula 8).

| Concentration of active | | |
|---|---|---|
| chlorine before buffering. | Postproduction buffering | Real-time buffering |
| Mg/L | concentration mg/L | concentration mg/L |
| 250 | 220 | 280 |
| 350 | 280 | 370 |
| 390 | 340 | 430 |

### Example: Method of Real-Time Buffering.

An aqueous solution of Na₂HPO₄ is prepared by diluting 500 g Na₂HPO₄ (anhydrous) in 10,000 mL pharmaceutical grade water (5% m/m solution). The solution pH is 8.80-9.30. The 5% m/m aqueous solution of disodium hydrogen orthophosphate should be made up for every batch of manufactured solution and any leftover buffer should be discarded after use.

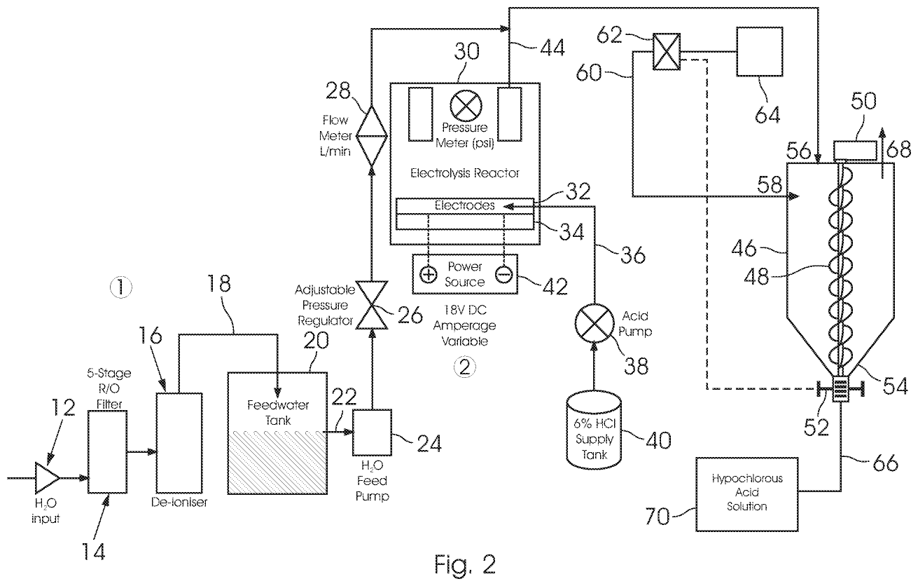

A pH-correction dosing pump (Tekna Evo APG603, controlled by K100PR proportional integral derivative pH controller for pH-correction www.seko.com) is connected to the pH correction chamber via the supplied pH-probe, which is placed at the distal end (outlet) of the dynamic mixer chamber (FIG. 2). The pump intake is placed in the buffering solution and the outlet is connected to the pH correction chamber at the proximal end of the dynamic mixer, next to the HOC/HCl solution inlet. The pH correction pump is set for correction to pH 5.40.

The removal of HCl [Reaction Formula 13] from the solution improves the stability of the HOCl in the solution.

The [Reaction Formula 8] demonstrates how the chemical reaction is forced towards the right, which favours the presence of HOCl over Cl₂.

CO₂+H₂O [symbol] HOCl+(HCl removed) [Reaction Formula 8]

### Oxidation-Reduction and the Role of Phosphoric Acid in Stabilizing the HOCl Solution.

Orthophosphate is an oxidation inhibitor (a substance that cannot donate electrons to an oxidant). Orthophosphate is available as phosphoric acid (H₃PO₄) and as its conjugate base, dihydrogen phosphate ion (H₂PO₄⁻). In an aqueous solution that contains H₃PO₄, the acid releases all H⁺ ions, however its ions reform with each other which again forms H₃PO₄. H₃PO₄ never fully dissociates in an aqueous solution and therefore an aqueous solution that contains H₃PO₄, always contains orthophosphate in the form of H₃PO₄ and H₂PO₄—.

Since the orthophosphate is in its highest oxidation state as phosphate ion, this ion cannot act as a reducing agent. It therefore protects HOCl, which is a strong oxidant, from being reduced.

Orthophosphate therefore prevents the break-down of HOCl by preventing oxidation-reduction reactions in the HOCl solution.

### Functions of Disodium Hydrogen Orthophosphate.

1. It acts as a real-time buffering agent of the acidic HOCl/HCl solution, thereby correcting the acidity level of the solution from pH 2.0 to pH 5.40 where all chlorine species exist as HOCl. The buffering process prevents the decomposition of HOCl into chlorate, by limiting the time that HOCl is present in a highly acidic environment. It also prevents the dissociation of HOCl into Cl₂ by maintaining the solution pH at the desired level pH 5.40.
2. The selective removal of HCl forces the reaction:

Cl₂+H₂O [symbol] HOCl+HCl [Reaction Formula 8]
to the right, which prevents the HOCl from reverting to Cl₂. This has a positive effect on the stability of HOCl.
3. The conjugate acid that forms from the buffering action of Na₂HPO₄ on HCl is phosphoric acid [Reaction Formula 13]. In an aqueous solution, the phosphoric acid, together with its dissociated conjugate base dihydrogen phosphate ion, both act as orthophosphate anti reducing agents, which prevents reduction of HOCl. This has a strong stabilizing effect on the HOCl solution.
4. H₃PO₄ serves as a buffer of the slightly acidic HOCl solution. This stabilizes the pH of the solution, preventing pH-shifts, which would favour dissociation and decomposition of HOCl in the solution.

Disodium hydrogen orthophosphate (Na₂HPO₄) principle functions therefore are to buffer the HOCl solution to the correct pH level and to stabilize the HOCl in the solution.

### Example: Method of Manufacturing Pharmaceutical Grade Hypochlorous Acid Solution

Having regard to FIG. 2.

### 1. Water Supply.

Water 12 is passed through a 5-stage reverse osmosis filter 14 followed by a deionizer 16 and any further process necessary to achieve conductivity of 0.15 to 0.21 μS/cm, producing a pharmaceutical grade water 18. The pharmaceutical grade water 18 is supplied to a feedwater tank 20.

A feed pump 24 with low volume high pressure characteristics (5.1 L/min, 170 psi (1172 kPa), 24 Volt DC, in this case a reverse osmosis water pump) supplies the water 22 via a pressure regulator 26 to the electrolysis chamber outlet. The flow rate of the water is measurable by a flow meter 28 (liquid flow meter [0.5 to 10 l/min][4-20 mA]). The rate of water supply to the electrolysis chamber outlet is regulated by adjusting the flow rate to 1.0 to 1.5 L/min, to provide production of 350 to 400 mg/L HOCl concentration. To achieve this flow rate, the water pressure should be 15 to 16 psi (103 to 110 kPa).

### 2. Electrolysis (Hydrolysis of HCl).

The electrolysis chamber 30 consists of multiple electrolytic plates 32-34 (iridium oxide-coated titanium) that are arranged for maximum HCl hydrolysis rate. It is a single chamber reactor, which is supplied with 6% platinum grade HCl solution 36 as the sole electrolyte.

Platinum grade HCl CAS Number 7647-01-0 has the following purity levels:

| Assay | min 32% |
|---|---|
| Arsenic (As) | 0.000001% |
| Bromide (Br) | 0.005% |
| Copper (Cu) | 0.000005% |
| Iron (Fe) | 0.00003% |
| Lead (Pb) | 0.00003% |
| Phosphate (PO4) | 0.00005% |
| Residue on ignition | 0.0005% |
| Sulphate (SO4) | 0.0001% |
| Zinc (Zn) | 0.000005% |

Concentrated platinum grade HCl 32% is diluted to 6% HCL by adding pharmacologically pure water to the concentrate before the electrolysis process. The diluted HCl 6% is kept in a storage tank 40 and delivered to the electrolysis chamber 30 by a peristaltic acid pump 38. This 6% HCl allows for a very high concentration of HOCl (1000's of mg/L) to be manufactured during electrolysis. Water is then added in a controlled manner to the concentrated HOCl before it emerges from the electrolysis chamber at the outlet 44, AFTER the electrolysis process. The flow rate of this water is regulated to determine the concentration of HOCl at the outlet 44, which is usually between 350 and 400 mg/L. The rate of acid delivery in the electrolysis chamber 30 is controlled via a PLC (2-4 mL/min). This volume of acid provides sufficient electrical conduction between the positive anode 34 and negative cathode 32 of the chamber for maximum HCl hydrolysis. Electrical supply 42 to the chamber is 18 v DC, at current strength of 3.20-3.40 amps. The electrical current strength provides for sufficient oxidation of Cl⁻ and the hydrolysis of Cl₂ at the anode to produce 350 mg/L to 400 mg/L of HOCl concentration after dilution by the input water.

It is important that the water does not pass through the electrolysis plates.

There is a significant risk of insufficient electrolysis if the feed water is allowed to pass through the electrolysis plates, which is what happens when pre-mixing of water and HCL is allowed to happen before electrolysis.

### 3. Mixer pH Correction Tank

As the produced HOCl solution has a highly acidic pH (pH 1.50 to 2.50), immediate buffering of the solution to pH 5.40 is required. Buffering takes place in a pH-correction chamber 46 that consist of the following components:
a) Auger screw 48 driven by a motor 50.
b) pH probe 52 in approximation to the outlet 54 of the mixer tank 46.
c) Inlet 56 to the mixer tank 46.
d) Inlet 58 for the disodium hydrogen orthophosphate aqueous solution 64 from the pH correction dosing pump 62, entering the mixer tank 46 next to the HOCl/HCl inlet 56.
e) Outlet 66 for pH corrected HOCl solution 54.
f) Drainage valve 68 at the top of the chamber 46, to release hydrogen and chlorine gas.

When the diluted HOCl/HCl product emerges from the electrolysis chamber, the pH of the solution is between 1.5 and 2.5. Because of the low pH, there is rapid decomposition into chlorate and dissociation into chlorine gas. Real-time buffering of this acidic solution therefore needs to be done.

Real time buffering refers to buffering within one second after emerging from the chamber. What is equally important, is that the product is buffered to the correct pH within one second.

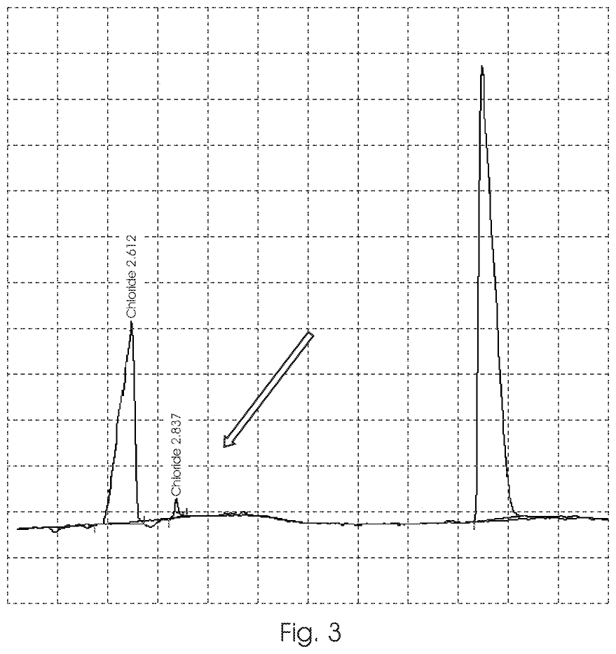

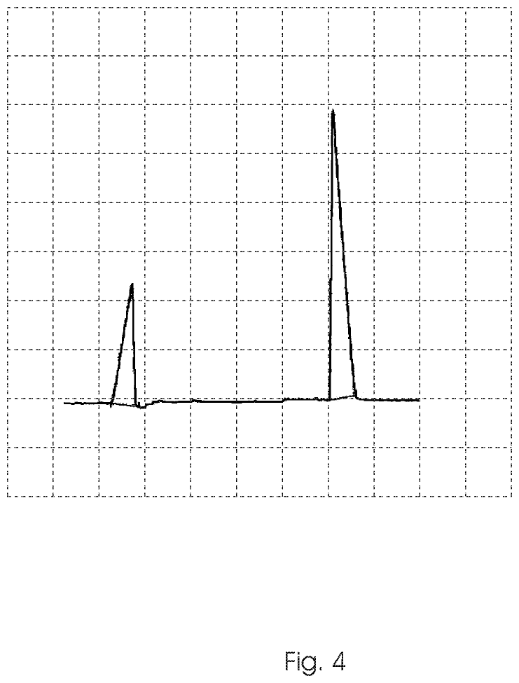

A toxic by-product chlorate (the formation of which passes through toxic chlorite production first), chlorine gas and hypochlorite forms due to the decomposition of hypochlorous acid at an acidic or alkaline pH environment. As the produced product is very acidic (pH less than 2.0), chlorate forms (see spectrophotometric analysis in FIG. 3 of post-production product, clearly showing a strong chloride signal “A”, toxic chlorate “B” present in the solution, and a low signal for hypochlorous acid “C” in FIG. 3. FIG. 4 shows a low signal for chloride “D”, no chlorate “E”, and a strong signal for hypochlorous acid “F”. The chlorate is not present with immediate buffering of the solution shown in FIG. 4.

### 4. pH Correction Pump.

The components of this pump 62 consist of:
i) Connection for pH probe 52
ii) Inlet/suction for receiving disodium hydrogen orthophosphate aqueous solution from a container 64.
iii) Outlet/positive pressure dosing pump for disodium hydrogen orthophosphate aqueous solution 60 to the dynamic mixer chamber 46.
iv) Proportional integral derivative controller panel for pump settings.
v) Pump settings: pump of alkaline solution, set pH value 5.40.

### 5. Product Collection Tank.

Product collection tank 70 for collecting hypochlorous acid solution 66 should be:
i) Sufficient volume to collect manufactured batch of HOCl solution.
ii) Closed off at the top to prevent contamination of the contents by liquids, gasses, or solids.
iii) Outflow valve to remove product.

### Example: Stability of a Hypochlorous Acid Solution Produced by the Method of the Present Invention

Chamber Conditions: 40° C./75% Relative Humidity.

| Test | Start Solution | 3 Months | 6 Months |
|---|---|---|---|
| Colour | Clear liquid | Clear liquid | Clear liquid |
| Cl Odour | Complies | Complies | Complies |
| pH | 5.50 | 5.35 | 5.20 |
| Moisture content | 114.9% | 115.3% | 111.5% |
| Assay: HOCL | 350 ppm | 329 ppm | 315 ppm |

### Examples of Clinical Applications of Pharmaceutical Grade HOCl Solution and Dosage of Use.

1. Skin care. Please note wetted gauze or foam refers to gauze or foam swabs wetted with pharmaceutical grade HOCl solution.
i) The control of inflammageing, the process of skin ageing. 250 mg/L skin spray, twice daily.

Pharmaceutical grade HOCl solution, when used twice per day, can control long-term low-grade inflammation. The low-grade inflammation that is responsible for skin ageing is termed inflammageing. The inflammageing has two consequences: ageing of the skin and chronic inflammatory skin conditions like rosaceae, acne, eczema, and folliculitis barbae (inflammation of the hair follicles of the beard-growing area). In our clinical experience pharmaceutical grade HOCl solution not only stops inflammageing but also has a positive effect on chronic inflammatory skin conditions.
ii) Rejuvenation skin care. 250 mg/L facial spray, twice daily application; long-term.

The appearance of an aged skin is evident as a having a sallow appearance, wrinkling, loss of tissue volume, hydration loss (dry skin) and loss of elasticity. Skin rejuvenation is effected by the long-term application of pharmaceutical grade HOCl solution. Rejuvenation is evident in the reversal of the age-compromised structural and functional components of the epidermis and dermis, leading to a more youthful looking skin that functions better. A more youthful looking colour, changing from being sallow looking to an even toned skin that is better hydrated and more elastic (less wrinkles), further improves the appearance of the skin.
iii) Aftercare for invasive skin treatments. 350 mg/L facial spray before invasive treatments to prevent infection. Repeat every 30 minutes until bedtime to control inflammation. Can be used as a glide during mesoderm needling treatments.

Aesthetic manipulation of the skin to improve the long-term appearance of the skin consists of invasive treatments like skin laser (ablative and non-ablative), skin needling (also called mesoderm treatment), injection of platelet rich plasma or dermal fillers which are designed to plump out wrinkles and areas where loss of volume in facial tissues create an impression of advanced age. Skin scraping, dermabrasion, the removal of cosmetic tattoo through laser and chemical means, also fall within this group. These treatments induce inflammation and has as an inherent risk of long-term inflammation (redness, swelling, pain and a raised temperature), which increase the risk of post-inflammatory hyperpigmentation (darkening of the skin). More so, the control of inflammation by the skin spray application of pharmaceutical grade HOCl solution, provides for the added advantage that people of colour can also be considered for skin manipulation treatments, as darker skin types have an increased risk of suffering from post-inflammatory hyperpigmentation after prolonged periods of inflammation. This increases the commercial potential of pharmaceutical grade HOCl solution in the aesthetics industry.

Apart from the control of inflammation, the HOCl solution also acts as a skin sterilizer before skin treatments are commenced. This avoids skin infection that may result from invasive skin treatments. Infection carries considerable risk of scarring of the skin. The use of pharmaceutical grade HOCl solution thereby acts as a risk mitigation device in the aesthetics industry.
iv) Skin Infection. 350 mg/L spray-on 3× per day or daily wetted gauze or foam dressings in severe cases.

Pharmaceutical grade HOCl solution, when manufactured according to the method described in this application, has efficacy as an antiseptic, not only against commonly occurring pathogens but also against multiple drug resistant bacteria, viruses, and fungi, including biofilm.

TABLE 1
Efficacy of pharmaceutical HOCl solution of the present invention against
MDR pathogens and viruses. SANS 51276: 2011: Chemical disinfectants
and antiseptics - Quantitative suspension test for the evaluation
of bactericidal activity of chemical disinfectants and antiseptics
used in food, industrial, domestic, and institutional areas.

| | Log- | Log- | Log- |
|---|---|---|---|
| Multiple drug resistant | reduction | reduction | reduction |
| Pathogens | 2 minutes | 5 minutes | 15 minutes |
| Extreme drug resistant | 5 | 6.0 | 7.0 |
| and drug sensitive | | | |
| Mycobacterium | | | |
| tuberculosis | | | |
| Pseudomonas | 4.6 | 5.2 | 5.5 |
| aeruginosa | | | |
| Acinetobacter | 7 | 7.0 | 7.0 |
| baumannii | | | |
| Carbapenem Resistant | 6.6 | 8.3 | 7.0 |
| Enterobacteriaceae | | | |
| (CRE), incl. OXA-48 | | | |
| Klebsiella pneumoniae | | | |
| Candida parapsilosis | 3.9 | 4.2 | 5.5 |
| MRSA | 4.5 | 5.5 | 6.0 |
| Human coronavirus | Complete | | |
| | inactivation | | |

Infection that may be treated with pharmaceutical grade HOCl solution, such as:
a) Fungal nail infections including yeast onychomycosis.
b) Athlete's foot (tinea pedis), fungal infection of the groin (tinea cruris).
c) Candidiasis of the oral cavity. Chronic mucocutaneous candidiasis, mucosal candidiasis, and cutaneous candidiasis. Folliculitis, caused by fungal infection.
d) Aspergillosis, cryptococcosis, histoplasmosis, blastomycosis, paracoccidioidomycosis, mucormycosis, oral geotrichosis, and fusariosis.
e) Erysipelas caused by streptococcus.
f) Bacterial folliculitis usually caused Pseudomonas aeruginosa.
g) Combinations of bacterial and fungal infections, usually with biofilm complicating local skin treatments, e.g., acne, folliculitis barbae and perioral dermatitis.
h) Furunculosis, carbuncle and impetigo caused by Staphylococcus aureus.
i) Erythrasma, caused by Corynebacterium minutissimum.
j) MRSA skin infections.
k) Nappy rash, omphalitis, and cradle cap infection in babies, as well as oral fungal infection.
l) Acne keloidaris nuchae, infection with inflammation that occur on the back of the neck, mostly in males.
m) Periorbital bacterial infection, which could contribute to dry eye syndrome.
n) Candida fungal infection of the breast nipple, especially during breast-feeding. This also prevents oral fungal infection in breast-fed babies.
v) Eye infection caused by bacteria, viruses, and fungi. These include but are not limited to meibomianitis, conjunctivitis, uveitis, and keratitis. 250 mg/L eye drops, 3-4 times per day.
vi) Sinusitis (allergic and infective). 350 mg/L nasal applicator, 2 times daily. Nasal application with a nasal applicator assists with the control of postnasal drip and symptoms and signs of sinus infection and allergies.
vii) Vaginal infection (fungal, bacterial). 350 mg/L, douche application, 30 ml 2-3 times per day.
viii) Leprosy, through the bactericidal action on Mycobacterium leprae, the cause of leprosy. 350 mg/L, daily wetted gauze, or foam dressings.
ix) Allergic and inflammatory skin conditions. 250 mg/L skin spray, once per day.
   i) Insect bites, allergic reaction to antigens (pollen, plant, or animal material).
   ii) Atopic dermatitis, psoriasis.
   iii) Sunburn
x) Wounds. 350 mg/L wetted gauze or foam dressings, 3-7 times per week. More frequent dressings provide faster healing.

Pharmaceutical grade HOCl solution has powerful anti-bacterial, anti-viral and anti-fungal properties. Furthermore, it destroys biofilm, which is secreted by pathogenic organisms as protection against onslaughts from the environment. Standard of care treatment include antiseptics and the application of antibiotics in the form of ointments and creams, which have largely become ineffective due to bacterial resistance to these products.

Pharmaceutical grade HOCl solution modulates inflammation, as described, and demonstrated as case studies in peer reviewed journals by the inventor. (Table 2).

TABLE 2
Publications attesting to the antipathogenic, anti-inflammatory and wound healing
efficacy of pharmaceutical grade HOCl solution of the present invention.

| | | Peer-reviewed | |
|---|---|---|---|
| Author | Title | Journal | Date |
| H Roos, B Kana, L | The use of | Wound Healing | 2021; 14(1): 21-24 |
| Naude | hypochlorous acid in | Southern Africa | |
| | an infected burn | | |
| | wound - a case | | |
| | study | | |
| H Roos, B Kana, J | The use of | South African | 2021; 4(1): 10-12 |
| Nel | hypochlorous acid in | Journal of Plastic | |
| | an OXA-48 multiple | and | |
| | drug-resistant | Reconstructive | |
| | Enterobacteriaceae- | Aesthetic Surgery | |
| | infected lower leg | and Burns | |
| | wound - a case | | |
| | study | | |
| H Roos, B Kana | The use of | Wound Healing | 2021; 14(2): 52-54 |
| | hypochlorous acid in | Southern Africa | |
| | an irradiation ulcer of | | |
| | the lower eyelid - a | | |
| | case study | | |
| H Roos, B Kana | The use of | South African | Accepted for |
| | hypochlorous acid in | Journal of Plastic | publication. |
| | the management of a | and | |
| | deep partial | Reconstructive | |
| | thickness burn - a | Aesthetic Surgery | |
| | case study. | and Burns | |
| H Roos, B Kana | Secondary intention | Wound Healing | 2022; 15(1): 10-12 |
| | wound healing using | Southern Africa | |
| | hypochlorous acid | | |
| | dressings: Case | | |
| | report. | | |
| H Roos | The use of | International | Volume 8, Issue 4, |
| | hypochlorous acid in | Journal of | December 2022, pp. |
| | treating diabetic foot | Biomedical | 53-56. doi: |
| | ulcer. | Engineering and | 10.11648/ |
| | | Clinical Science | j.ijbecs.20220804.13 |

Types of wounds that can be treated by pharmaceutical grade HOCl solution of the present invention.
a) Acute wounds (traumatic, surgical and burn wounds), to prevent infection and the formation of biofilm, which is the cause of wounds becoming chronic. 350 mg/L wound irrigation in the case of penetrating injuries.
b) Chronic wounds, including venous ulcers, diabetic foot ulcers, arterial ulcers, rheumatoid ulcers, pressure sores or any wound that is not healing through conventional treatment methods. 350 mg/L wetted gauze or foam dressings daily.
c) Orthopaedic septic joint replacement wound irrigation to prevent periprosthetic joint infection and revisional surgery. 350 mg/L wound irrigation daily.
d) Infected burn wounds. 350 mg/L dressings with paraffin gauze or foam, covered with wetted gauze or foam, daily.
e) Acute and chronic episiotomy wounds. 350 mg/L spray-on application, 4 times per day.
xi) Lung pathology

Pharmaceutical grade HOCl solutions has been found suitable for inhalation. Indications for inhalation include infective and inflammatory lung conditions, which may be acute or chronic. Inhalation is through ultrasonic/mechanical misting devices. 350 mg/L, 3-5 ml solution per nebulizing session, 3×per day for active disease, once daily as prophylaxis.
a) Infective lung conditions, bacterial, including acute bacterial pneumonia, chronic lung conditions due to primary and secondary bacterial infection e.g., Pseudomonas aeruginosa. Infective lung conditions with a strong inflammatory component like tuberculosis (drug sensitive as well as drug resistant strains of Mycobacterium tuberculosis), and inflammatory lung affliction in HIV patients are further indications.
b) Infective lung conditions, viral. Pharmaceutical grade HOCl solution has been proven in vitro to kill viruses. Our clinical experience confirms that viral lung disease with associated lung inflammation e.g., severe acute respiratory syndrome (SARS) can be treated by inhaling the solution.
c) Infective lung conditions, fungal. Candida and mucormycosis (black fungus) can be treated with pharmaceutical grade HOCl solution. Respiratory syncytial virus lung infections can be treated with pharmaceutical grade HOCl solution.
d) Treatment of degenerative lung conditions to prevent secondary infection. Conditions include cystic fibrosis, chronic obstructive pulmonary disease (COPD) and pulmonary fibrosis.
e) Treatment of degenerative lung conditions to reduce scarring (COPD, atelectasis, post infective lung fibrosis).
f) Treatment of inflammatory lung conditions (asthma, HIV), pneumonitis.
xii) Treatment of inflammatory bowel disease and ulcers of the perianal area.
a) Ulcerative colitis can be improved with colonic irrigation using pharmaceutical grade HOCl solution. 350 mg/L, 50-100 ml colonic washes.
b) Chancroid, genital herpes, granuloma inguinale and lymphogranuloma venereum. 350 mg/L spray-on application, 4 times per day.

1. A method of manufacturing pharmaceutical grade hypochlorous acid, the method comprises:
a) adding platinum grade hydrochloric acid with a concentration of 5 to 7%, to an in inlet of an electrolysis chamber;
b) executing an electrolysis process to produce hypochlorous acid;
c) supplying water with a conductivity of 0.15 to 0.25 μS/cm to mix with and produce hypochlorous acid with a pH of 1.5 to 2.5;
d) adding a buffering agent to the hypochlorous acid produced at step c) to adjust the pH of the hypochlorous acid to between 4.0 and 5.8, producing a pharmaceutical grade hypochlorous acid.

2. The method claimed in claim 1, wherein the hypochlorous acid produced at step c) has a concentration of 350 to 400 mg/L.

3. The method claimed in claim 1, wherein the buffering agent is added to the hypochlorous acid in less than 2 seconds of the production of the hydrochloric acid.

4.-5. (canceled)

6. The method claimed in claim 1, wherein the platinum grade hydrochloric acid has a concentration of 6%.

7. The method claimed in claim 1, wherein the buffering agent is an orthophosphate buffer in an aqueous solution.

8. The method claimed in claim 7, wherein the buffering agent is platinum grade disodium hydrogen orthophosphate or dipotassium hydrogen orthophosphate buffer in an aqueous solution at a 1% to 10% m/m concentration.

9. The method claimed in claim 1, wherein the pH of the hypochlorous acid in step d) is about 5.40.

10. The method claimed in claim 1, wherein the hydrochloric acid is added to the electrolysis chamber at step c) at a rate of between 1-5 mL/min.

11.-12. (canceled)

13. The method claimed in claim 1, wherein the water is added to the electrolysis chamber at step c) at a rate of between 0.3 to 2 L/min.

14.-15. (canceled)

16. The method claimed in claim 1, wherein the water supplied to the hypochlorous acid at step c) has a conductivity of 0.18 to 0.22 μS/cm.

17. (canceled)

18. The method claimed in claim 1, wherein, at step c), the water is supplied to an outlet of the electrolysis chamber.

19. The method claimed in claim 18, wherein the electrolysis chamber is a single chamber reactor.

20. The method claimed in claim 19, wherein the electrolysis chamber comprises of multiple electrolytic plates.

21. The method claimed in claim 20, wherein the electrolytic plates are iridium oxide-coated titanium.

22. The method claimed in claim 1, wherein the buffering agent is added to the hypochlorous acid in a pH correction mixing chamber, wherein the pH correction chamber optionally comprises an augur screw mixer for mixing the hypochlorous acid and the buffering agent.

23. The method claimed in claim 22, wherein the pH correction chamber consists of an entry point for hypochlorous acid and an entry point for the buffering agent.

24. The method claimed in claim 23, wherein the pH correction chamber comprises a dosing pump that automatically buffers the hypochlorous acid to the adjusted pH.

25. The method claimed in claim 24, wherein the mixing chamber includes a pH measuring electrode for real-time pH-measurement, and wherein the pH electrode communicates with a controller that adjusts buffer dosing at a dosing pump to deliver a pre-determined product pH.

26.-29. (canceled)

30. A pharmaceutical grade hypochlorous acid produced by the method claimed in claim 1.

31. A pharmaceutical grade hypochlorous acid claimed in claim 30, for use in a method for treating skin infections, inflammation, fungi, bacteria or viruses, wherein the method comprises administering a therapeutically effective amount of said pharmaceutical grade hypochlorous acid to a subject.

32. A cosmetic method of rejuvenating the skin comprising administering the pharmaceutical grade hypochlorous acid claimed in claim 30 to a subject.
